(19) United States
(12) Patent Application Publication
(10) Pub. No.: US 2026/0157879 A1
(43) Pub. Date: Jun. 11, 2026

(54) SQUEEZABLE CONTAINERS WITH INTEGRATED VALVE FOR MULTIDOSE DISPENSING

(71) Applicant: Alcon Inc., Fribourg (CH)

(72) Inventors: Brian C. Patterson, Burleson, TX (US); Allison Renee Campolo, Euless, TX (US); Stephen Paul Shannon, Arlington, TX (US); Reed Allen Pifer, Willow Park, TX (US); Monica Jane Crary, Fort Worth, TX (US)

(21) Appl. No.: 19/412,228

(22) Filed: Dec. 8, 2025

Related U.S. Application Data
(60) Provisional application No. 63/729,698, filed on Dec. 9, 2024.

(51) Int. Cl. A61F 9/00

(52) U.S. Cl. CPC A61F 9/0008

(57) ABSTRACT

In some embodiments, a system for dispensing a fluid includes a dispensing plug and a squeezable container for receiving the dispensing plug and storing the fluid. The dispensing plug includes a one-way valve for dispensing the fluid. The squeezable container includes a squeezable wall, an opening for dispensing the fluid that is sealed airtight by the dispensing plug, an air-return hole in the squeezable wall, and an air-return valve that is permeable to air and impermeable to the fluid and that closes the air-return hole. The fluid leaves the squeezable container through the one-way valve of the dispensing plug in response to exerting a pressure onto the squeezable wall, and air enters the squeezable container through the air-return valve upon expansion of the squeezable wall in response to releasing the pressure onto the squeezable wall.

### INTRODUCTION

Many pathologies of the eye are treated by direct application of fluids to the eye, such as liquid eye drops. For example, conjunctivitis is treated by directly applying eye drops containing antibiotics. Dry eyes and glaucoma are also treated using eye drops. In general, eye drops and other treatment fluids can be used as lubricants for relieving discomfort and dryness of the eyes, as well as delivery vehicles for therapeutic substances.

Eye drops, as well as other treatment fluids, are typically stored in a squeezable container with a built-in dispensing device. To apply a treatment fluid to a patient's eye, the squeezable container is manually compressed by a user to dispense the treatment fluid through the dispensing device. However, there is a risk of microbial ingress to the container.

### SUMMARY

Embodiments of the present disclosure provide squeezable containers, such as multidose preservative free (MDPF) bottles, and systems including the same, for dispensing fluids (including liquids, gels, solutions, emulsions, and suspensions). The squeezable containers of the present discloser facilitate the reduction and/or prevention of microbial ingress into the containers while also enabling equalization of pressure, and therefore, restoration of the shape of the containers, after dispensing of fluids.

In some embodiments, a system for dispensing fluids is provided, the system including: a dispensing plug comprising a one-way valve for dispensing the fluid, and a squeezable container for receiving the dispensing plug and storing the fluid. The squeezable container includes a squeezable wall, an opening for dispensing the fluid that is sealed airtight by the dispensing plug, an air-return hole in the squeezable wall, and an air-return valve that closes the air-return hole, wherein the air-return valve is permeable to air and impermeable to the fluid, wherein the fluid leaves the squeezable container through the one-way valve of the dispensing plug in response to exerting a pressure onto the squeezable wall, and wherein air enters the squeezable container through the air-return valve upon expansion of the squeezable wall in response to releasing the pressure onto the squeezable wall.

In some embodiments, a squeezable container for storing a fluid in a fluid-dispensing system having a dispensing plug with a one-way valve for dispensing the fluid is provided, the squeezable container comprising: a squeezable wall, an opening for receiving the dispensing plug, an air-return hole in the squeezable wall, and an air-return valve that closes the air-return hole, wherein the air-return valve is permeable to air and impermeable to the fluid.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 is an exploded view of a system that may be utilized for dispensing fluids, according to some embodiments of the present disclosure.

FIG. 2A is a schematic cross-sectional side view of an example dispensing plug in a closed state, according to some embodiments of the present disclosure.

FIG. 2B is a schematic cross-sectional side view of the example dispensing plug of FIG. 2A in an open state, according to some embodiments of the present disclosure.

FIG. 3A is a schematic partial cross-sectional side view of a squeezable container with an air-return hole and an air-return valve situated in the body portion of the squeezable container, according to some embodiments of the present disclosure.

FIG. 3B is a schematic partial cross-sectional side view of a squeezable container with an air-return hole and an air-return valve situated in the neck portion of the squeezable container, according to some embodiments of the present disclosure.

FIG. 3C is a schematic partial cross-sectional side view of a squeezable container with an air-return hole and an air-return valve situated in the shoulder portion of the squeezable container, according to some embodiments of the present disclosure.

FIG. 3D is a schematic partial cross-sectional side view of a squeezable container with an air-return hole and an air-return valve situated in the base portion of the squeezable container, according to some embodiments of the present disclosure.

FIG. 4 is a schematic partial cross-sectional side view of a squeeze tube with an air-return hole and an air-return valve situated in the body portion of the squeeze tube, according to some embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments of the present disclosure provide a squeezable container with an integrated valve mechanism for dispensing a fluid, such as a low viscosity fluid. The fluid may generally include liquids, gels, solutions, emulsions, and suspensions, including ophthalmic fluids such as lubricating eye drops, medicated/prescription eye drops, ophthalmic gels, ophthalmic ointments, ophthalmic stains, and other preservative free ocular solutions.

Examples of squeezable containers include eye drop dispensers, droptainers, drop bottles, squeeze bottles, dropping bottles, and squeeze tubes. To actuate the dispensing of fluids contained within a squeezable container, portions of a body of the squeezable container may be compressed or squeezed by a user, pump, or other actuation mechanism (including electronically controlled mechanisms). This compression increases the pressure within the squeezable container and/or compresses the fluids therein, thereby causing the fluids to be expelled through an opening, or outlet, of the squeezable container.

In some embodiments, the present squeezable container includes a valve mechanism integrated into the container that facilitates the equalization of pressure after dispensing the fluid, and thus, restoration of the shape of the container. In some embodiments, the squeezable container can be incorporated into a system for dispensing the fluid, which can further include a dispensing plug for dispensing the fluid from the squeezable container.

In some embodiments, the valve mechanism of the squeezable container reduces, and in some examples, prevents microbial ingress when dispensing fluids from the aforementioned squeezable container, while allowing passive ingress of air. Accordingly, embodiments of the present disclosure relate to a squeezable container that effectively resists microbial contamination, allows passive air entry, and facilities restoration of the shape of the container, thereby providing improved fluid preservation as well as usage and storage of the devices.

Examples will now be described relative to the Drawings.

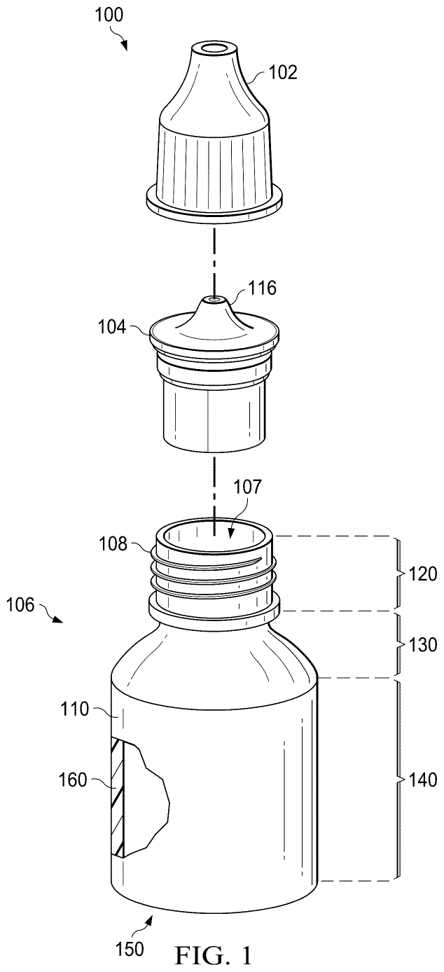

FIG. 1 is an exploded view of a system 100 for dispensing a fluid. The system 100 includes a dispensing plug 104, and a squeezable container 106 for receiving the dispensing plug 104 and storing the fluid. The term “squeezable container” as used hereinafter refers to a container with flexible walls that can be easily squeezed or compressed when pressure forces are acting on the outside walls of the container. Thereby, the container is deformable and changes, at least temporarily, its original shape.

The squeezable container 106 includes a squeezable wall 160 and an opening 107 for dispensing the fluid. The squeezable container 106 can be, for example, a bottle, a tube, or droptainer. In the illustrated embodiment, the squeezable container 106 is shown as a squeeze bottle 110 having a tubular or cylindrical shape.

In some embodiments, the squeezable container 106 is made of a material that can be deformed in response to exerting a pressure force onto the squeezable wall 160. If desired, the material can be selected such that the squeezable container 106 returns to its original shape in response to releasing the pressure force on the squeezable wall 160. As an example, the squeezable container 106 can be made of any material that has an elastic recovery including elastomers such as rubber, polyurethane, ethylene-propylene monomer, etc. As another example, the squeezable container 106 can be made of a shape memory material, including shape-memory polymers or shape-memory alloys, etc.

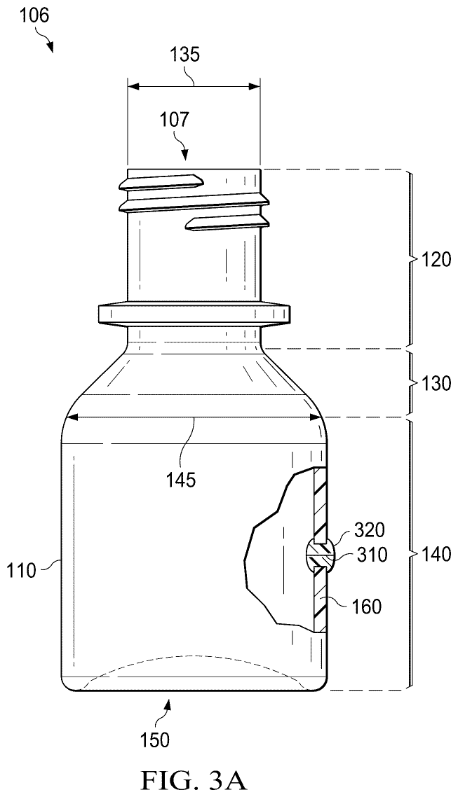

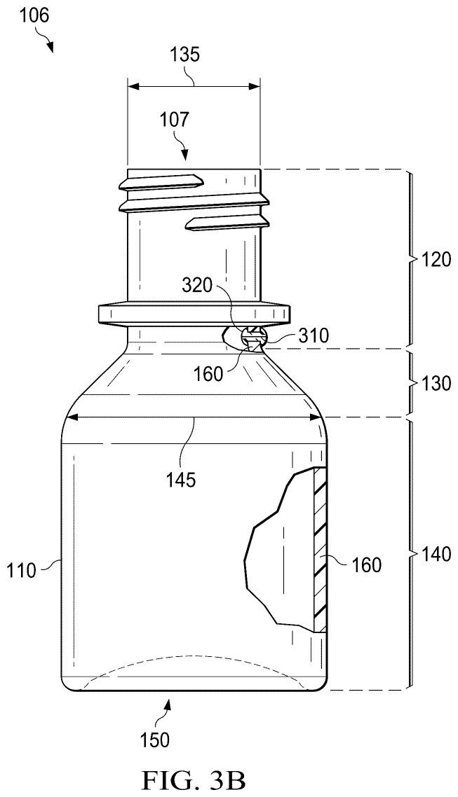

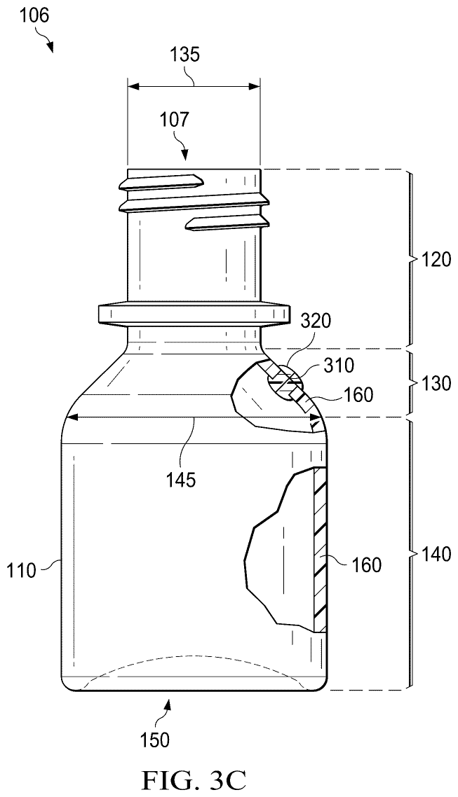

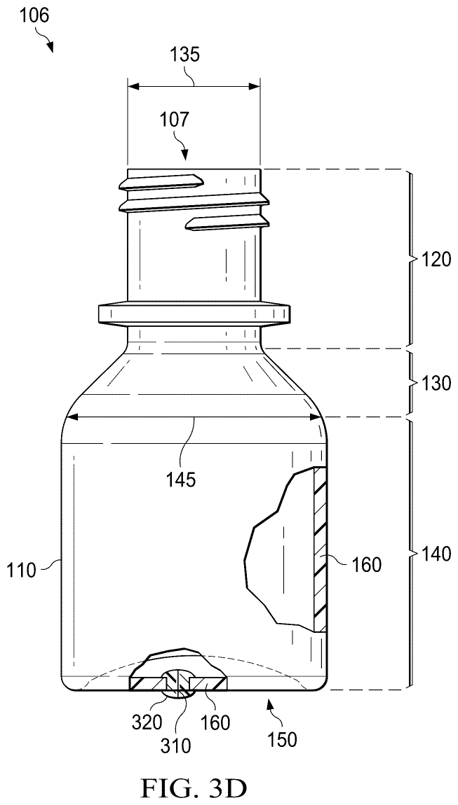

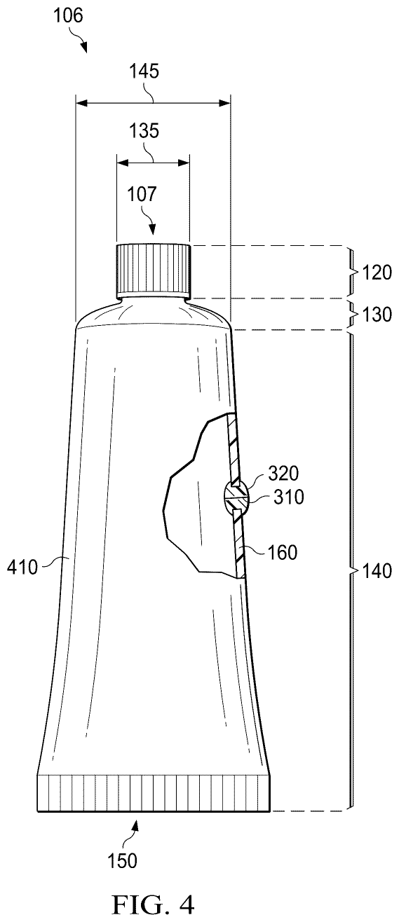

To equalize the negative pressure inside the squeezable container 106 that results from exerting a pressure force onto the squeezable wall 160 and subsequently releasing the pressure force, the squeezable container 106 includes an air-return hole in the squeezable wall 160. An air-return valve is disposed within the air-return hole and provides a barrier to prevent microbial ingress into the squeezable container 106 and to prevent the fluid from leaking through the air-return hole. For this purpose, the air-return valve is permeable to air (and/or other gases) and impermeable to fluids. Different embodiments of such an air-return hole and air-return valve are shown in FIG. 3A, FIG. 3B, FIG. 3C, FIG. 3D, and FIG. 4.

The squeezable container 106 can include a neck portion 120 with a neck diameter 135 next to the opening 107, a body portion 140 with a body diameter 145 that is greater than the neck diameter 135, and a base portion 150 that is situated opposite the opening 107 and next to the body portion 140. In some embodiments, the squeezable container 106 includes a shoulder portion 130 that is situated between the neck portion 120 and the body portion 140.

It should be appreciated that the shape shown relative to the squeeze bottle 110 is merely illustrative. In various embodiments, the squeeze bottle 110 can have any suitable geometry, shape, and/or size including, for example, tapered shapes, ovaloid shapes, and/or the like. In some embodiments, the squeezable container 106 includes an external thread 108 for attachment of a cap 102.

In some embodiments, the system 100 includes the cap 102. The cap 102 can be attached to the squeezable container 106 to seal the system 100 for storage, for example, between uses, or for long-term storage. The cap 102 can be placed over the dispensing plug 104 to seal off, or block, a tip 116 of the dispensing plug 104 from the external environment, thereby obstructing or preventing microbes or other contaminants from entering the system 100 during storage, as well as preventing fluids from unintentionally spilling out of system 100.

In some embodiments, the cap 102 is configured to secure onto the squeezable container 106 or the dispensing plug 104 via a threaded connection. In such embodiments, an inner surface of the cap 102 can include one or more threads or grooves disposed on an inner surface thereof and configured to mate with one or more threads or grooves 108 disposed on an outer surface of the squeezable container 106 or on an outer surface of the dispensing plug 104. In other embodiments, however, the cap 102 can be secured onto the squeezable container 106 or the dispensing plug 104 via a snap fit, friction fit, clasp, or the like. As shown in FIG. 1, the cap 102 can removably attach to the neck portion 120 of the squeezable container 106 via a threaded connection.

In some embodiments, the cap 102 includes an additional plug. The additional plug can be configured to slide into the tip 116 of the dispensing plug 104 and function as a stopper to further prevent contaminants from entering the system 100 and/or to prevent the fluid from unintentionally spilling out. Further, the additional plug can function to reduce a volume of fluid contained in the dispensing plug 104 during storage by filling at least some of the space in the channel of the dispensing plug 104 through which the fluids are passed, which reduces the chances of microbial growth in these passages that can contaminate a next dispensed dose of fluid.

For this purpose, the additional plug of the cap 102 can include an elongated body shaped and sized to substantially occupy, or fill, the space in the channel of the dispensing plug 104 when the cap 102 secures the dispensing plug 104. In some scenarios, it can be beneficial to have the tip 116 of the dispensing plug 104 secured/closed by the additional plug, even when the pressure in the squeezable container 106 increases to a point that would normally cause the fluid to be dispensed.

The dispensing plug 104 is placed inside the opening 107 of the squeezable container 106 such that the opening 107 is sealed airtight by the dispensing plug 104. For example, in some embodiments, the dispensing plug 104 is compression-or friction-fit inside the opening 107 of the squeezable container 106. In some embodiments, the dispensing plug 104 can be placed over the opening 107 and attached to the squeezable container 106 via an outer surface of at least a part of the neck portion 120. In these embodiments, the cap 102 can be attached to the dispensing plug 104 instead of, or in addition to, the squeezable container 106.

The dispensing plug 104 can be made, for example, wholly or partially of silicone or another suitable polymer.

In the illustrated embodiment, fluid from the squeezable container 106 can be transmitted from the opening 107 of the squeezable container 106, into the dispensing plug 104, by applying compression force against the outer surface of the squeezable wall 160 of the squeezable container 106, which then increases the fluid pressure within the squeezable container 106, to force fluids out the tip 116.

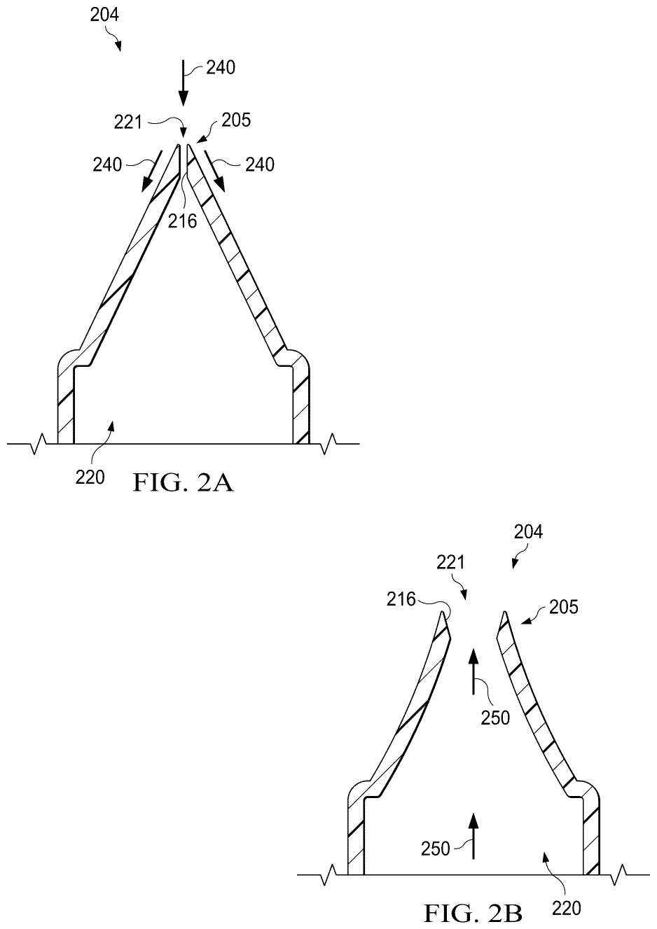

FIG. 2A is a schematic cross-sectional side view of an example dispensing plug 204 in a closed state. FIG. 2B is a schematic cross-sectional side view of the example dispensing plug 204 of FIG. 2A in an open state.

With reference to FIGS. 2A-B collectively, the dispensing plug 204 may correspond, for example, to dispensing plug 104 of FIG. 1. The dispensing plug 204 has a tip 216 similar to the tip 116 of FIG. 1. The dispensing plug 204 further includes an outlet 221 at the tip 216 and an inlet 220 that is, for example, connected with the opening 107 of the squeezable container 106 of FIG. 1 such that the opening is sealed airtight by the dispensing plug 204. The inlet 220 corresponds to an ingress point of fluids into the dispensing plug 204. The outlet 221 corresponds to an egress point of fluids out the tip 216.

The dispensing plug 204 includes a one-way valve 205 for dispensing fluids. The one-way valve 205 allows the fluids to flow from the inlet 220 to the outlet 221 and through the tip 216 while preventing gases, solids, or liquids other than the dispensing fluids to enter the tip 216 and/or flow from the outlet 221 to the inlet 220.

The dispensing plug 204 can be made wholly or partially of silicone or another polymer, and can be selected to optimize the sealing characteristics. In some embodiments, the one-way valve 205 is formed of an elastic and hydrophobic material, while the remaining portions of the dispensing plug 204 are formed of a rigid and hydrophobic material. Examples of suitable rigid materials include polyethylene such as a low-density polyethylene (LDPE), a high-density polyethylene (HDPE), a cyclic olefin copolymer (COC), a polyetherimide (PEI), a polytetrafluoroethylene (PTFE), a polyoxymethylene (POM), and combinations thereof. Examples of suitable elastic materials include silicone or thermoplastic elastomers. In further embodiments, the one-way valve 205 is formed of a rigid hydrophobic material, while the remaining portions of the dispensing plug 204 are formed of an elastic and hydrophobic material. In still further embodiments, both of the one-way valve 205 and the remaining portions of the dispensing plug 204 are formed of a rigid material or an elastic material.

The valve 205 can be any type of a one-way valve suitable to allow egress of the fluid from the inlet 220 through the outlet 221 and the tip 216, and to further prevent ingress of gases, liquids, and/or solids through the tip 216, into the outlet 221, and to the inlet 220. For example, the valve 205 can include a duckbill valve, a flapper valve, an umbrella valve, or any other suitable type of check valve.

In some embodiments, the one-way valve 205 exerts a force that causes the dispensing plug 204 to be in a closed state as shown in FIG. 2A. For descriptive purposes, this force is referred to herein as a biasing force. The biasing force facilitates sealing of the dispensing plug 204 unless, or until, the biasing force is overcome by an opposing force such as, for example, fluid pressure on the inlet 220. The fluid pressure can be caused by compression force(s) against the squeezable wall of a squeezable container, such as the squeezable wall 110 of the squeezable container 106 of FIG. 1.

When the opposing force overcomes the biasing force, the one-way valve 205 at least partially opens a fluid path 250 for the fluid from the inlet 220 to the outlet as shown in FIG. 2B, thereby allowing the fluid to flow from the inlet 220 to the outlet 221 and to be dispensed out the tip 216 in proportion to an amount by which the opposing force exceeds the biasing force.

When the opposing force no longer overcomes the biasing force, for example, as a result of a decrease in, or cessation of, the compression force, the biasing force, by virtue of its continuous or constant nature, immediately causes the one-way valve 205 to again seal the fluid path 250 between the inlet 220 and the outlet 221. In this way, the one-way valve 205 can seal the dispensing plug 204 whenever fluids are not being dispensed, thereby preventing microbial ingress 240 as shown in FIG. 2A. Accordingly, in various embodiments, microbial ingress, for example, to a container such as the squeezable container 106 of system 100 of FIG. 1, can likewise be reduced or prevented.

However, since there is no air-return flow through the dispensing plug 104 into the squeezable container 106 of FIG. 1, a negative pressure is building up inside the system 100 leading to a lasting deformation (in squeezed form) of the squeezable container 106 and/or the dispensing plug 104. To prevent the lasting deformation of the squeezable container 106 and to ensure that the squeezable container 106 returns to its original shape after dispensing of the fluid, the squeezable container 106 includes an air-return hole in the squeezable wall 160. To prevent leakage of the fluid through the air-return hole and to reduce/prevent microbial ingress into the squeezable container 106, the squeezable container 106 includes an air-return valve that seals the air-return hole. The air-return valve is permeable to air and impermeable to the fluid such that air enters the squeezable container 106 through the air-return valve upon expansion of the squeezable wall 160 in response to releasing the pressure onto the squeezable wall 160 after dispensing of the fluid.

FIGS. 3A, 3B, 3C, and 3D show schematic partial cross-sectional side views of a squeezable container 106 with respective air-return holes 310 and corresponding air-return valve 320. FIG. 3A shows the air-return hole 310 and the corresponding air-return valve 320 situated in the body portion 140 of the squeezable container 106, FIG. 3B shows the air-return hole 310 and the corresponding air-return valve 320 situated in the neck portion 120 of the squeezable container 106, FIG. 3C shows the air-return hole 310 and the corresponding air-return valve 320 situated in the shoulder portion 130 of the squeezable container 106, and FIG. 3D shows the air-return hole 310 and the corresponding air-return valve 320 situated in the base portion 150 of the squeezable container 106.

In some embodiments, the squeezable container 106 can include more than one air-return hole 310 with a corresponding air-return valve 320. The one or more additional air-return holes and the corresponding air-return valves may be situated in the same portion, or in other portions, of the squeezable container 106.

As an example, the squeezable container 106 can have two or more air-return holes with corresponding air-return valves that are all situated in the same portion of the squeezable container 106 (e.g., all air-return holes and air-return valves are situated in the neck portion 120, all air-return holes and air-return valves are situated in the shoulder portion 130, all air-return holes and air-return valves are situated in the body portion 140, or all air-return holes and air-return valves are situated in the base portion 150 of the squeezable container 106).

As another example, the squeezable container 106 can have at least two air-return holes with corresponding air-return valves in at least two different portions of the squeezable container 106, such as two air-return holes with corresponding air-return valves in the body portion 140 of the squeezable container 106 and one air-return hole in the shoulder portion 130 of the squeezable container 106.

As yet another example, the squeezable container 106 can have at least one air-return hole with a corresponding air-return valve in each portion of the squeezable container 106. Thus, the squeezable container 106 can have at least one air-return hole 310 and associated air-return valve 320 in the neck portion 120, at least one air-return hole 310 and associated air-return valve 320 in the shoulder portion 130, at least one air-return hole 310 and associated air-return valve 320 in the body portion 140, and at least one air-return hole 310 and associated air-return valve 320 in the base portion 150.

The air-return valve 320 that closes the air-return hole of FIG. 3A, 3B, 3C, or 3D is permeable to air and impermeable to the fluid that is stored inside the squeezable container 106, such that the fluid only leaves the squeezable container 106 through the opening 107 in response to exerting a pressure force onto the squeezable walls 110 of the squeezable container 106, and air enters the squeezable container 106 through the air-return valve 320 upon expansion of the squeezable wall 160 in response to releasing the pressure onto the squeezable wall 160.

The number and size of the air-return holes 310 and the corresponding air-return valves 320 are dimensioned such that the squeezable container 106 returns to its original shape within a predetermined time in response to releasing the pressure on the squeezable wall 160. In some embodiments, the number and size of the air-return holes 310 and the corresponding air-return valves 320 are dimensioned such that the squeezable container 106 returns within, at most, five seconds to its original shape in response to releasing the pressure onto the squeezable wall 160. In other embodiments, the number and size of the air-return holes 310 and the corresponding air-return valves 320 are dimensioned such that the squeezable container 106 returns within, at most, two or three seconds to its original shape in response to releasing the pressure onto the squeezable wall 160. In yet other embodiments, the number and size of the air-return holes 310 and the corresponding air-return valves 320 are dimensioned such that the squeezable container 106 returns within, at most, one second to its original shape in response to releasing the pressure onto the squeezable wall 160.

In some embodiments, the air-return valve 320 includes an air-permeable membrane. The air-permeable membrane can be made of any suitable material that is permeable to air and impermeable to the fluid stored inside the squeezable container 106. If desired, the air-permeable membrane can include polytetrafluoroethylene (PTFE), polydimethylsiloxane (PDMS), polyolefin pulp, polyurethane engineered to be breathable, microporous polypropylene (PP), silicone, nonwoven fabrics with hydrophobic coatings such as fluoropolymers, and/or other suitable polymeric materials.

In some embodiments, the air-return valve 320 has the same dimensions of the air-return hole 310 such that the air-return valve 320 fits within the air-return hole 310 and does not extend over any portions of an interior and/or outer surface of the squeezable bottle 106. In other embodiments, the air-return valve 320 is disposed over the air-return hole 310 and over a portion of the interior and/or outer surface of the squeezable bottle 106, such that the portion of the interior and/or outer surface of the squeezable bottle 106 is covered by the air-return valve 320. For example, a label that includes an air-permeable membrane can be attached to the outside of the body portion 140 of the squeezable container 106 and thereby cover one or more air-return holes 310 in the body portion 140 of the squeezable container 106, as well as portions of the outside of the squeezable wall 160, to form the corresponding air-return valve(s) 320. In yet other embodiments, and as shown in FIGS. 3A, 3B, 3C, and 3D, the air-return valve 320 can fill the air-return hole 310 and, in addition, cover a portion of an interior and/or outer surface of the squeezable bottle 106 in the vicinity of the air-return hole 310.

The squeezable container 106 can include a squeeze bottle 110 as shown in FIGS. 3A, 3B, 3C, and 3D. In some embodiments, the squeezable container 106 includes a squeeze tube or other type of fluid container.

FIG. 4 is a schematic partial cross-sectional side view of a squeeze tube 410 with an air-return hole 310 and an air-return valve 320. In the embodiments shown, the squeeze tube 410 has a neck portion 120, a shoulder portion 130, a body portion 140, and a base portion 150. As shown in FIG. 4, the air-return hole 310 and the air-return valve 320 are situated in the body portion 140 of the squeeze tube 410.

However, the air-return hole 310 and the air-return valve 320 can be situated instead, or in addition, in one or more other portions of the squeeze tube 410. In some embodiments, more than one air-return hole 310 with a corresponding air-return valve 320 can be situated in a same or in different portions of the squeeze tube 410. As an example, the air-return hole 310 and the air-return valve 320 can be situated in the neck portion 120 or shoulder portion 130 of the squeeze tube 410.

Generally, the various components of the system 106 or 406 can be individually fabricated by any suitable molding techniques, such as by injection molding and/or liquid silicone rubber injection molding, and/or three-dimensional (3D) printing techniques, and thereafter press fit, snap fit, friction fit, or otherwise coupled together during assembly. In some embodiments, however, one or more components of the system 106 can be monolithically molded and/or 3D printed. Similarly, one or more of the components of the squeezable container 106 can be fabricated by molding techniques, such as by injection molding and/or liquid silicone rubber injection molding, and/or 3D printing techniques, and thereafter press fit, snap fit, friction fit, or otherwise coupled together during assembly. In some embodiments, however, the squeezable container 106 is monolithically molded and/or 3D printed.

In summary, embodiments of the present disclosure provide systems and apparatuses for dispensing fluids (including, without limitation, liquids, gels, solutions, emulsions, suspensions, and the like) from a squeezable container, while maintaining the shape of the container after dispensing of the fluid and preventing microbial ingress into the container. In some embodiments, the systems and apparatuses of the present disclosure include a dispensing plug with a one-way valve and a squeezable container that together prevent microbial ingress when dispensing fluids from the aforementioned squeezable container, while still allowing passive ingress of air. Accordingly, embodiments of the present disclosure relate to systems and apparatuses that effectively resist microbial contamination of the fluid therein and allow passive air entry, thereby facilitating improved fluid preservation and storage.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited in the claims using the phrase “means for” or, in the case of a method claim, the element is recited using the phrase “step for.”

While various examples have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described examples.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘including’ as used herein is synonymous with ‘including,’ ‘containing,’ or ‘comprising,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the described subject matter, but instead as merely intended to highlight alternative or additional features that may or may not be utilized in a particular example of the described subject matter. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the described subject matter to the specific examples and examples described herein, but rather to also cover all modification and alternatives coming with the true scope and spirit of the described subject matter.

What is claimed is:

1. A system for dispensing a fluid, comprising:
a dispensing plug comprising a one-way valve for dispensing the fluid; and
a squeezable container for receiving the dispensing plug and storing the fluid, comprising:
a squeezable wall;
an opening for dispensing the fluid that is sealed airtight by the dispensing plug;
an air-return hole disposed through the squeezable wall; and
an air-return valve disposed in and sealing the air-return hole, wherein the air-return valve is permeable to air and impermeable to the fluid, wherein the fluid exits the squeezable container through the one-way valve of the dispensing plug in response to exerting a pressure onto the squeezable wall, and wherein air enters the squeezable container through the air-return valve upon expansion of the squeezable wall in response to releasing the pressure onto the squeezable wall.

2. The system of claim 1, wherein the air-return valve comprises an air-permeable membrane.

3. The system of claim 2, wherein the air-permeable membrane comprises silicone, polytetrafluoroethylene (PTFE), polydimethylsiloxane (PDMS), polyolefin pulp, or combinations thereof.

4. The system of claim 1, wherein the squeezable container further comprises:
a neck portion with a neck diameter next to the opening;
a body portion with a body diameter that is greater than the neck diameter; and
a base portion that is disposed opposite the opening and next to the body portion.

5. The system of claim 4, wherein the air-return hole is disposed in the base portion.

6. The system of claim 4, wherein the air-return hole is disposed in the neck portion.

7. The system of claim 4, wherein the squeezable container further comprises:
a shoulder portion disposed between the neck portion and the body portion, wherein the air-return hole is disposed in the shoulder portion.

8. The system of claim 4, wherein the air-return hole is disposed in the body portion.

9. The system of claim 8, further comprising:
a label comprising an air-permeable membrane attached to the body portion and covering the air-return hole in the body portion to form the air-return valve.

10. The system of claim 1, wherein the dispensing plug is compression-fit inside the opening of the squeezable container.

11. The system of claim 10, wherein the squeezable container comprises an external thread in a neck portion next to the opening, further comprising:
a cap with an internal thread configured to be screwed onto the external thread of the squeezable container.

12. The system of claim 1, wherein the squeezable container comprises a squeeze tube or squeeze bottle.

13. A squeezable container for storing a fluid in a fluid-dispensing system having a dispensing plug with a one-way valve for dispensing the fluid, comprising:
a squeezable wall;
an opening for receiving the dispensing plug;
an air-return hole traversing a thickness of the squeezable wall; and
an air-return valve that closes the air-return hole, wherein the air-return valve is permeable to air and impermeable to the fluid.

14. The squeezable container of claim 13, wherein the air-return valve comprises an air-permeable membrane that comprises silicone, polytetrafluoroethylene (PTFE), polydimethylsiloxane (PDMS), polyolefin pulp, or combinations thereof.

15. The squeezable container of claim 13, further comprising:
a neck portion with a neck diameter next to the opening;
a body portion with a body diameter that is greater than the neck diameter; and
a base portion that is disposed opposite the opening and next to the body portion, wherein the air-return hole is disposed in the neck portion, the body portion, and/or the base portion.
